(19) United States
(12) Patent Application Publication
(10) Pub. No.: US 2019/0166122 A1
(43) Pub. Date: May 30, 2019

(54) AUTHENTICATION TERMINAL

(71) Applicant: AISIN SEIKI KABUSHIKI KAISHA, Kariya-shi (JP)

(72) Inventors: Kenta MOCHIZUKI, Yokohama-shi (JP); Yosuke Aoyagi, Koganei-shi (JP)

(73) Assignee: AISIN SEIKI KABUSHIKI KAISHA, Kariya-shi (JP)

(21) Appl. No.: 16/199,365

(22) Filed: Nov. 26, 2018

(30) Foreign Application Priority Data
Nov. 27, 2017 (JP) 2017-227091

(51) Int. Cl. H04L 29/06; G06F 21/32; A61B 5/117; A61B 5/22; A61B 5/024; A61B 5/00; A61B 5/0408

(52) U.S. Cl. CPC H04L 63/0861; G06F 21/32; A61B 5/117; A61B 5/0408; A61B 5/02438; A61B 5/6801; A61B 5/224

(57) ABSTRACT

An authentication terminal includes: a casing that is worn on a body of a target person; first and second electrodes that are provided in the casing and measure a measurement potential as biological information; a light emitting unit that is provided in the casing and emits measurement light for measuring a muscle potential to the body of the target person; a light receiving unit that receives the measurement light and outputs muscle potential information which corresponds to the muscle potential; and a terminal-side control unit that outputs authentication information which is generated based on the muscle potential that is indicated by the muscle potential information, and on a cardiac potential that is calculated from the measurement potential between the first electrode and the second electrode to another device.

### CROSS REFERENCE TO RELATED APPLICATIONS

This application is based on and claims priority under 35 U.S.C. § 119 to Japanese Patent Application 2017-227091, filed on Nov. 27, 2017, the entire contents of which are incorporated herein by reference.

### TECHNICAL FIELD

This disclosure relates to an authentication terminal.

### BACKGROUND DISCUSSION

An authentication terminal is known that authenticates a target person based on a biological signal of a human body. For example, the authentication terminal measures a cardiac potential of the target person as the biological signal of the human body using a pair of electrodes, compares data that is registered in advance and the cardiac potential, and determines whether or not the target person is a registrant that is registered.

However, because a potential that is measured using the pair of electrodes also includes a muscle potential and the like other than the cardiac potential, the precision of the authentication by the above-described authentication terminal is low. Therefore, a technology is disclosed that removes noise, such as the muscle potential that is included in the measured potential and the like, using a band pass filter and increases the precision of the authentication.

Examples of the related art include JP 2014-239737A and JP 2017-51611A.

However, the above-described device uniformly removes a component of a specific frequency that is included in the cardiac potential, as the muscle potential, using the band pass filter, not depending on a state of the target person. Because of this, the removal of the muscle potential is insufficient and one portion of the cardiac potential other than the muscle potential is also removed. As a result, there is a problem in that the precision of the authentication of the registrant, which uses the cardiac potential, is not sufficient in the above-described device.

Thus, a need exists for an authentication terminal which is not susceptible to the drawback mentioned above.

### SUMMARY

An authentication terminal according to an aspect of this disclosure includes: a casing that is worn on a body of a target person; first and second electrodes that are provided in the casing and measure a measurement potential as biological information; a light emitting unit that is provided in the casing and emits measurement light for measuring a muscle potential to the body of the target person; a light receiving unit that receives the measurement light and outputs muscle potential information which corresponds to the muscle potential; and a terminal-side control unit that outputs authentication information which is generated based on the muscle potential that is indicated by the muscle potential information, and on a cardiac potential that is calculated from the measurement potential between the first electrode and the second electrode to another device.

### BRIEF DESCRIPTION OF THE DRAWINGS

The foregoing and additional features and characteristics of this disclosure will become more apparent from the following detailed description considered with the reference to the accompanying drawings, wherein:

FIG. 1 is a perspective diagram of an authentication terminal according to an embodiment;

FIG. 2 is a plan-view diagram of the outside of the authentication terminal in a worn state;

FIG. 3 is a plan-view diagram of the inside of the authentication terminal;

FIG. 4 is a functional block diagram for describing a function of an authentication system that includes the authentication terminal;

FIG. 5 is a graph for describing a waveform of a cardiac potential of an ordinary person.

FIG. 6 is a table illustrating an example of a registrant database;

FIG. 7 is a flowchart illustrating an example of each of the authentication processing and the control processing that are performed by the authentication system; and

FIG. 8 is a flowchart illustrating an example of registration processing that is performed by the authentication terminal.

### DETAILED DESCRIPTION

The same constituent elements in the following exemplary embodiment and the like are given the same reference numeral, and a description thereof is not properly repeated.

### Embodiment

An authentication terminal 10 according to an embodiment is a bracelet type device that is worn on the vicinity of a wrist or the like of an authentication target person (hereinafter referred to as a target person OP). The authentication terminal 10 calculates a cardiac potential that results from converting an electrical change in the heart of the target person OP into an electric potential, and, based on the cardiac potential, determines whether or not the target person OP that wears the authentication terminal 10 is a registrant that is registered. The authentication terminal 10 outputs a result of the determination as authentication information. An example of the authentication terminal 10 is a smart key that authenticates the target person OP in order to unlock a door lock of a vehicle such as a motor car or cause a driving source, such as an engine, to start.

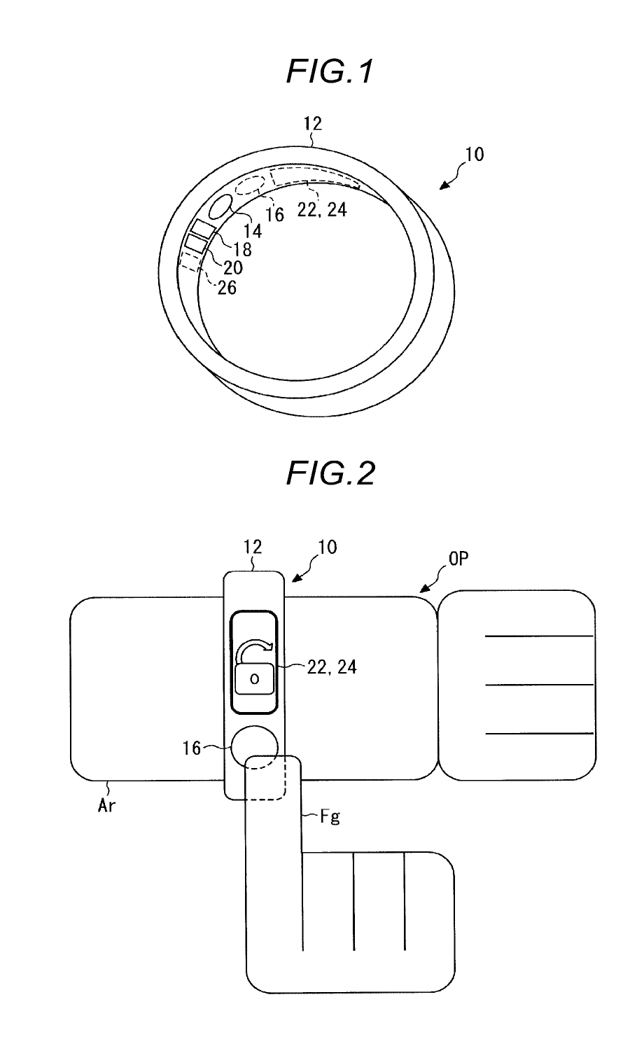

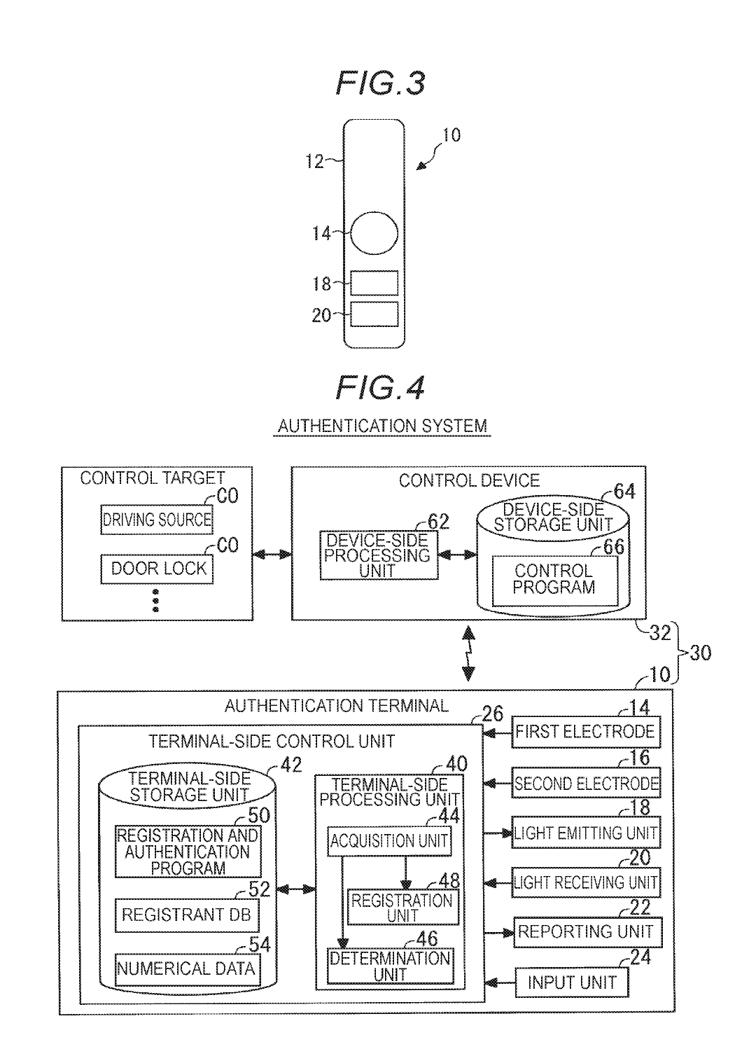

FIG. 1 is a perspective diagram of the authentication terminal 10 according to the embodiment. FIG. 2 is a plan-view diagram of the outside of the authentication terminal 10 in a worn state. FIG. 3 is a plan-view diagram of the inside of the authentication terminal 10. As illustrated in FIGS. 1, 2, and 3, the authentication terminal 10 includes a casing 12, a first electrode 14, a second electrode 16, a light emitting unit 18, a light receiving unit 20, a reporting unit 22, an input unit 24, and a terminal-side control unit 26.

The casing 12, for example, is configured to be in the shape of a ring that has a hole in the center. An internal diameter of the casing 12 may be such that the authentication terminal 10 can be worn on the wrist of the target person OP who wants to wear the authentication terminal 10, and is not particularly limited. The casing 12, for example, is configured with an insulating material such as resin.

The first electrode 14 is configured with a conductive material such as metal. The first electrode 14 is electrically connected to the terminal-side control unit 26. The first electrode 14 is provided inside of the casing 12.

The second electrode 16 is configured with a conduction material such as metal. The second electrode 16 is electrically connected to the terminal-side control unit 26. The second electrode 16 is provided outside of the casing 12. It is preferable that in a circumferential direction of the casing 12, the second electrode 16 is provided in the vicinity of the first electrode 14 or in the same position as the first electrode 14. Accordingly, when the second electrode 16 is pressed with a finger Fg of the target person OP, the first electrode 14 can make contact with an arm Ar of the target person OP.

Both the first electrode 14 and the second electrode 16 make contact one portion of a body of the target person OP and thus measure a measurement potential as biological information. Specifically, when the finger Fg of one hand (for example, the right hand) of the target person OP makes contact with and presses the second electrode 16, the first electrode 14 makes contact with a surface in the vicinity of the wrist, of the arm Ar (for example, the left arm) that is opposite the hand with which the second electrode 16 is in contact. Accordingly, the first electrode 14 and the second electrode 16 measure a potential between the first electrode 14 and the second electrode 16, as the measurement potential. At this point, because the heart of the target person OP is present between the first electrode 14 and the second electrode 16, the measurement potential includes the cardiac potential that is one piece of biological information. As described above, the first electrode 14 makes contact with the arm Ar in the vicinity of the wrist and the second electrode 16 makes contact with the finger Fg or the like of the opposite hand. Because of this, a difference between a path from the first electrode 14 to the heart and a path from the second electrode 16 to the heart increases. As a result, the authentication terminal 10 can improve the accuracy of the calculation of the cardiac potential. The first electrode 14 and the second electrode 16 output measurement potential information indicating the measurement potential, to the terminal-side control unit 26. Moreover, the measurement potential information may be the measurement potential itself.

The light emitting unit 18 emits light (hereinafter referred to as measurement light) for measuring a muscle potential to the body of the target person OP. The light emitting unit 18, for example, is a light emitting diode (LED) that emits blue light, as the measurement light, that has a wavelength of 435 nm to 480 nm and has difficulty in penetrating into the body. The light emitting unit 18 is provided inside of the casing 12. Therefore, the light emitting unit 18 emits the measurement light to arm Ar of the target person OP inside of the casing 12. It is preferable that the light emitting unit 18 is provided in the vicinity of the first electrode 14. It is preferable that, for example, in the circumferential direction of the casing 12, the light emitting unit 18 is positioned at or below an angle of 90° with respect to the first electrode 14.

The light receiving unit 20 receives the measurement light that is emitted by the light emitting unit 18 and is reflected by the target person OP, and outputs muscle potential information that is generated as a result of converting the measurement light into an electrical signal, to the terminal-side control unit 26. At this point, because the measurement light corresponds to the muscle potential, the muscle potential information that results from converting the measurement light into the electrical signal corresponds to the muscle potential of the target person OP. The light receiving unit 20 is a photo diode or a phototransistor. The light receiving unit 20 is provided inside of the casing 12. It is preferable that the light receiving unit 20 is provided in the vicinity of the first electrode 14 and the light emitting unit 18. It is preferable that for example, in the circumferential direction of the casing 12, the light receiving unit 20 is positioned at or below an angle of 90° with respect to the first electrode 14 and the light emitting unit 18. Accordingly, the light receiving unit 20 almost faces an area to which the measurement light is emitted by the light emitting unit 18, and thus can efficiently receive the measurement light.

The reporting unit 22 reports report information, which is acquired from the terminal-side control unit 26, to the target person OP. The reporting unit 22, for example, includes any one of a display device, such as a liquid crystal display, a speaker that possibly outputs audio, and a vibration member, such as an actuator that causes the authentication terminal 10 to vibrate. The reporting unit 22, for example, reports a result of authentication that determines whether or not the registrant is present based on the cardiac potential, information on the target person OP, such as a health state relating to the cardiac potential, information relating to a worn position of the authentication terminal 10, and the like, to the target person OP, using any one of an image, audio, and vibration.

The input unit 24 receives an instruction or the like from the target person OP and outputs the instruction or the like, which is received, to the terminal-side control unit 26. The input unit 24, for example, is provided on a surface of the reporting unit 22. The input unit 24, for example, is a touch panel that possibly transmits light. Accordingly, the input unit 24 transmits an image that is displayed on the reporting unit 22. The input unit 24, for example, receives an instruction for authentication or registration from the target person OP, and outputs corresponding instruction information to the terminal-side control unit 26.

The terminal-side control unit 26 administers general control of the authentication terminal 10. The terminal-side control unit 26, for example, is a computer.

FIG. 4 is a functional block diagram for describing a function of an authentication system 30 that includes the authentication terminal 10. As illustrated in FIG. 4, the authentication system 30 includes the authentication terminal 10 and a control device 32.

The terminal-side control unit 26 has a terminal-side processing unit 40 and a terminal-side storage unit 42.

The terminal-side processing unit 40, for example, is a processor such as a central processing unit (CPU). The terminal-side processing unit 40 has an acquisition unit 44, a registration unit 48, and a determination unit 46. The terminal-side processing unit 40, for example, reads a registration and authentication program 50 that is stored in the terminal-side storage unit 42, and thus may function as the acquisition unit 44, the registration unit 48, and the determination unit 46. Moreover, one portion or all portions of each of the acquisition unit 44, the registration unit 48, and the determination unit 46 may be configured with an application specific integrated circuit (ASIC), a field-programmable gate array (FPGA), or the like.

The acquisition unit 44 acquires various pieces of information, and outputs the acquires various pieces of information to the registration unit 48 or the determination unit 46. For example, the acquisition unit 44 acquires the measurement potential information that includes information relating to the cardiac potential of the target person OP from the first electrode 14 and the second electrode 16 that are in contact with the target person OP. The acquisition unit 44 outputs the acquired measurement potential information to the registration unit 48 and the determination unit 46. The acquisition unit 44 acquisition the muscle potential information that includes information relating to the muscle potential, from the light receiving unit 20. The acquisition unit 44 outputs the acquired muscle potential information to the registration unit 48 and the determination unit 46. The acquisition unit 44 acquires pieces of instruction information that are input by the target person OP, such as an authentication instruction and a registration instruction, from the input unit 24. The acquisition unit 44 outputs the acquired instruction information to the registration unit 48.

The determination unit 46 generates the authentication information based on the muscle potential that is indicated by the muscle potential information, and on the cardiac potential of the target person OP, which is calculated from the measurement potential that is output by the electrodes 14 and 16, and outputs the generated authentication information to the control device 32. For example, the determination unit 46 may determine whether or not the target person OP is registered in a registrant database 52, based on the cardiac potential of the target person OP, may generate a result of the determination as the authentication information, and may output the authentication information. Specifically, the determination unit 46 uniformly removes the muscle potential that is indicated by the muscle potential information, from the measurement potential that is indicated by the measurement potential information, and calculates the cardiac potential. Because the measurement potential includes the cardiac potential and the muscle potential, a potential difference that results from removing the muscle potential from the measurement potential is almost the cardiac potential. At this point, the determination unit 46 may correct amplitude (that is, a maximum value) of the muscle potential that is indicated by the muscle potential information, may remove the corrected muscle potential from the measurement potential, and may output the cardiac potential. Specifically, the determination unit 46 may multiply the amplitude of the muscle potential by a correction coefficient, may make a scale adjustment, and thus may correct the muscle potential. The correction coefficient may be stored, as one portion of numerical data 54, in the terminal-side storage unit 42. The determination unit 46 extracts one, or two or more feature values, which are determined in advance, from the calculated cardiac potential. In a case where the extracted feature value of the cardiac potential is registered in cardiac potential information in the registrant database 52, the determination unit 46 determines that the target person OP is a registrant. On the other hand, in a case where the feature value of the cardiac potential is not registered in the cardiac potential information in the registrant database 52, the determination unit 46 determines that the target person OP is not a registrant. When it is determined that the target person OP is a registrant, the determination unit 46 outputs a result of the determination, as the authentication information, to the control device 32.

Based on the muscle potential that is indicated by the muscle potential information which is acquired from the acquisition unit 44, and on determination muscle potential that is registered in advance by the target person OP, the determination unit 46 may determine whether or not the worn position of the authentication terminal 10 is suitable. Accordingly, a true value of muscle potential for determination is set with worn-position information registered by the target person OP as a true value. For example, the determination unit 46 may extract the amplitude of the muscle potential as a feature value of the muscle potential, and may determine whether or not the worn position is suitable, depending on whether or not the amplitude is higher than the muscle potential for determination that is determined in advance. The muscle potential for determination, for example, is stored, as one portion of the numerical data 54, in the terminal-side storage unit 42. Specifically, in a case where the amplitude of the muscle potential is higher than determination muscle potential for determination that is determined in advance, the determination unit 46 may determine that the worn position of the authentication terminal 10 is suitable. On the other hand, in a case where the amplitude of the muscle potential is lower than the muscle potential for determination that is determined in advance, the determination unit 46 may determine that the worn position of the authentication terminal 10 is unsuitable. In a case where the muscle potential is equal to the muscle potential for determination that is determined in advance, the determination unit 46 may determine that the worn position of the authentication terminal 10 is suitable, or may determine that the worn position of the authentication terminal 10 is unsuitable. This determination may be performed by proper setting. Moreover, it is preferable that the determination unit 46 makes a determination of the worn position with the amplitude, as is, of the muscle potential, without correcting the muscle potential that is indicated by the muscle potential information, that is, without multiplying the muscle potential by the correction coefficient. Furthermore, the determination unit 46 may extract a frequency of the muscle potential that changes over time, which is indicated by the muscle potential information, as a feature value, and, with a difference between the frequency and a frequency for determination that is determined in advance, may determine whether or not the worn position of the authentication terminal 10 is suitable. Additionally, with both of the amplitude of the muscle potential and the frequency of the muscle potential as feature values, the determination unit 46 may determine whether or not the worn position of the authentication terminal 10 is suitable. In a case where it is determined that the worn position is suitable, as described above, based on the cardiac potential, the determination unit 46 may determine whether or not the target person OP is a registrant and may generate the authentication information. On the other hand, in a case where it is determined that the worn position is unsuitable, the determination unit 46 may cause the reporting unit 22 to report to the target person OP that the worn position is unsuitable, using any one of an image, audio, and vibration.

The determination unit 46 may provide information relating to a state of the target person OP based on the calculated cardiac potential. For example, the determination unit 46 may cause the reporting unit 22 to report a health state relating to a pulse wave or the like of the target person OP, which is calculated from the cardiac potential that is registered in the registrant database 52, or may output the health state together with the authentication information to the control device 32.

The registration unit 48 registers one, or two or more feature values that are extracted from cardiac potential of a new target person OP, as pieces of cardiac potential information, in the registrant database 52 that is stored in the terminal-side storage unit 42. For example, the registration unit 48 calculates a potential difference that results from removing the muscle potential that is indicated by the muscle potential information from the measurement potential that is indicated by the measurement potential information, as cardiac potential. At this point, the registration unit 48 may calculate the cardiac potential using muscle potential for correction, which results from correcting the muscle potential that is indicated by the muscle potential information. For example, the registration unit 48 multiplies the muscle potential, which is indicated by the muscle potential information, by the correction coefficient, makes a scale adjustment, and calculates the muscle potential for correction. The registration unit 48 extracts one, or two or more feature values that are indicated by the calculated cardiac potential, as pieces of cardiac potential information, and registers the cardiac potential information in the registrant database 52, in a state of being associated with a registrant ID indicating a registrant.

The terminal-side storage unit 42 has storage devices, such as a Random Access Memory (RAM), a Read Only Memory (ROM), a Solid State Drive (SSD), and a Hard Disk Driver (HDD), that are connected to the terminal-side processing unit 40. The terminal-side storage unit 42 may be a storage device that is provided outside and is connected to the terminal-side processing unit 40 through a network. Stored in the terminal-side storage unit 42 are a program that is executed by the terminal-side control unit 26, data necessary to execute the program, and data that is generated by executing the program. For example, the registration and authentication program 50 that is executed by the terminal-side control unit 26 is stored in the terminal-side storage unit 42. Moreover, instead of the registration and authentication program 50, a registration program for registering a registrant and an authentication program for authenticating the target person OP may be individually stored in the terminal-side storage unit 42. The registrant database 52 and the numerical data 54 are stored, as pieces of data necessary for executing the registration and authentication program 50, in the terminal-side storage unit 42. The registrant database 52, for example, is a database that results from associating a registrant ID indicating a registrant and cardiac potential information on the registrant with each other. Pieces of numerical data 54 include a correction coefficient that is used to execute the registration and authentication program 50, a numerical value for determination, a threshold, and the like. The measurement potential information, the muscle potential information, and the like on the target person OP are temporarily stored in the terminal-side storage unit 42.

The control device 32, for example, is a computer, such as an Electronic Control Unit (ECU), that is provided in a vehicle, such as a motor car. The control device 32 is configured in such a manner that information is possibly transmitted and received through communication such as wireless communication with the authentication terminal 10. Based on the authentication information on the target person OP, which is received from the authentication terminal 10, the control device 32 controls a control target CO of a vehicle. The control target CO, for example, is a driving source, such as an engine or a motor, a door lock of a vehicle, an air conditioner, or the like. The control device 32 has an apparatus-side processing unit 62 and an apparatus-side storage unit 64.

The apparatus-side processing unit 62, for example, is a hardware processor such as a CPU. The apparatus-side processing unit 62 reads a control program 66 that is stored in the apparatus-side storage unit 64, and thus performs various controls. For example, when acquiring the authentication information that the target person OP is a registrant, from the authentication terminal 10, the apparatus-side processing unit 62 unlocks a door lock of a vehicle, and causes a driving source, such as an engine, to start, or approves starting.

The apparatus-side storage unit 64 has storage devices, such as a RAM, a ROM, an SSD, and a HDD. The apparatus-side storage unit 64 may be a storage device that is provided outside and is connected to the apparatus-side processing unit 62 through a network. Stored in the apparatus-side storage unit 64 are the control program 66 that is executed by the apparatus-side processing unit 62, data that is necessary to execute the control program 66, and the like.

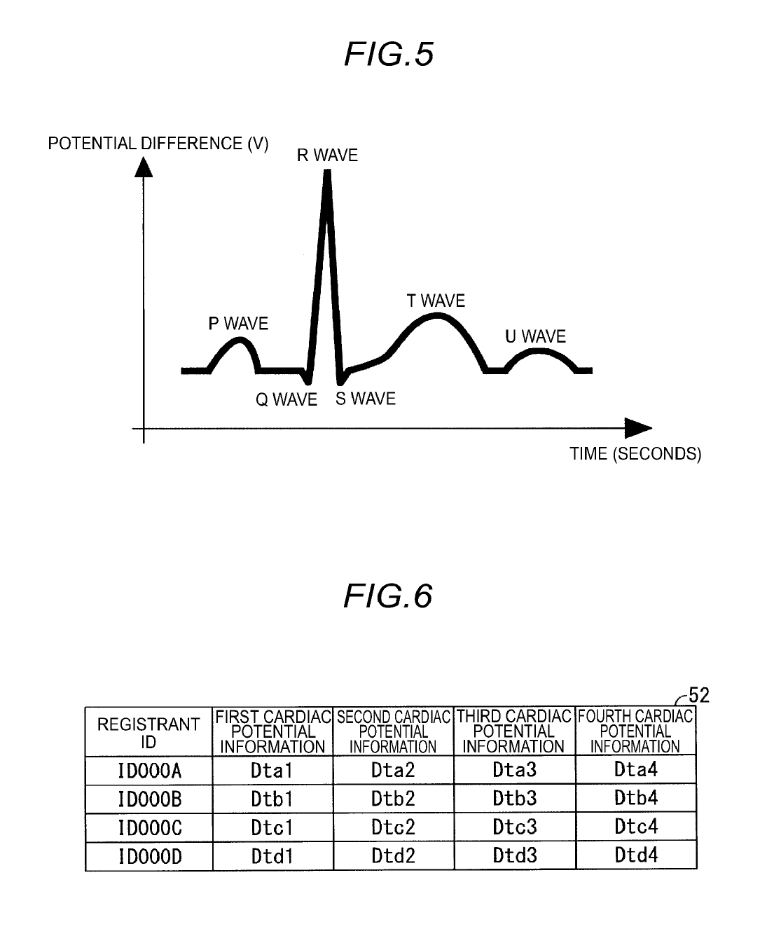

FIG. 5 is a graph for describing a waveform of cardiac potential of an ordinary person. As illustrated in FIG. 5, the waveform (that is, an electrocardiogram) of the cardiac potential of the ordinary person includes a plurality of peaks. The plurality of peaks, for example, include a P wave, a Q wave, an R wave, an S wave, a T wave, a U wave, and the like.

FIG. 6 is a table illustrating an example of the registrant database 52. As illustrated in FIG. 6, the registrant database 52 includes one, or two or more registrant IDs, and each registrant ID is associated with one piece of, or two or more pieces of cardiac potential information. The two or more pieces of cardiac potential information may be feature values of different waveforms of cardiac potential. For example, a feature value of a waveform of cardiac potential may be any one of amplitude (that is, potential difference), a wave width, and an inter-wave interval of each of a P wave, a Q wave, an R wave, an S wave, a T wave, and U wave. The wave width is time from a starting point to an ending point of each of the P wave, the Q wave, the R wave, the S wave, the T wave, and the U wave. The starting and ending points are time at which potential exceeds a reference value that is determined in advance and time at which potential is at or below the reference value, respectively. The inter-wave interval is a time interval between two starting points or ending points of each of the P wave, the Q wave, the R wave, the S wave, the T wave, and the U wave.

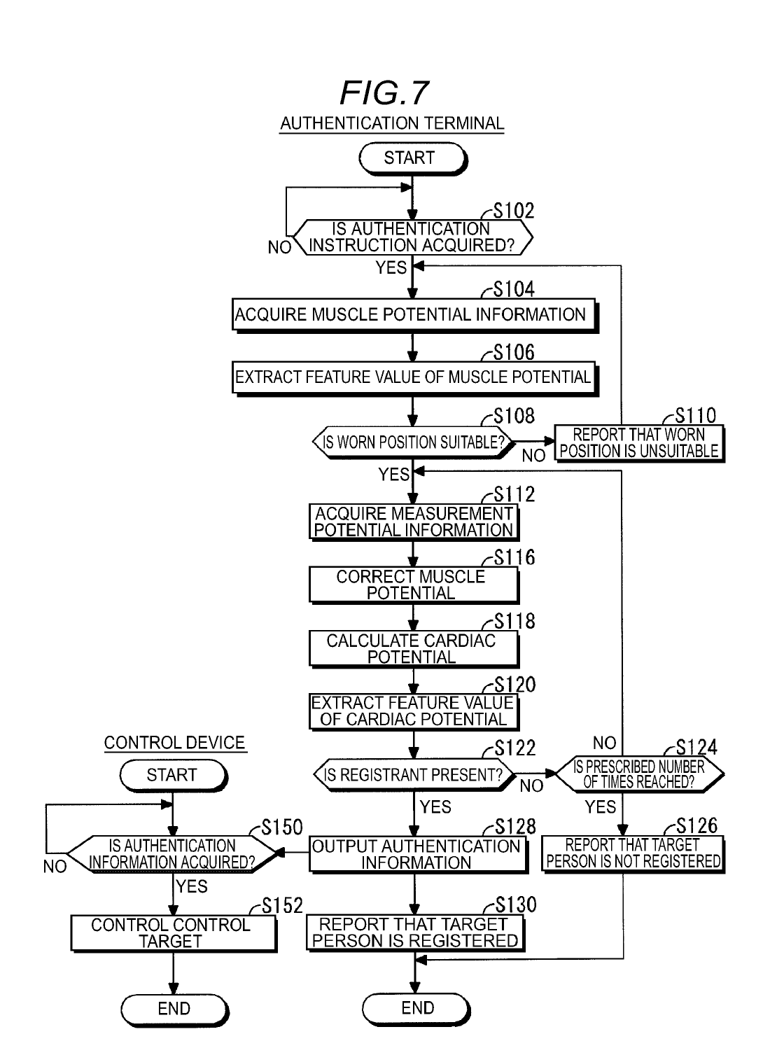

FIG. 7 is a flowchart illustrating an example of each of the authentication processing and the control processing that are performed by the authentication system 30. A flowchart on the right side of FIG. 7 is a flowchart for the authentication processing that is performed by the authentication terminal 10. A flowchart on the left side of FIG. 7 is a flowchart for the control processing that is performed by the control device 32. The authentication terminal 10 reads the registration and authentication program 50 and thus performs the authentication processing. The control device 32 reads the control program 66, and thus performs the control processing.

As illustrated in FIG. 7, when the authentication processing starts, the acquisition unit 44 determines whether or not the authentication instruction is acquired (S102). For example, when the target person OP inputs the authentication instruction using the input unit 24, the acquisition unit 44 may determine that the authentication instruction is acquired. Furthermore, when the finger Fg of the target person OP makes contact with the second electrode 16 and thus the measurement potential changes, the acquisition unit 44 may determine that authentication instruction is acquired. The acquisition unit 44 repeats Step S102 until the authentication instruction is acquired, and is in a standby state (No in S102).

On the other hand, when it is determined that the authentication instruction is acquired (Yes in S102), the acquisition unit 44 causes the light emitting unit 18 to emit the measurement light, acquires the muscle potential information from the light receiving unit 20 that receives the measurement light which is reflected by the target person OP, and outputs the acquired muscle potential information to the determination unit 46 (S104). The determination unit 46 extracts a feature value, such as the amplitude or the frequency of the muscle potential, from the muscle potential information (S106). Based on the feature value of the muscle potential and the muscle potential for determination, the determination unit 46 determines whether the worn position of the authentication terminal 10 is suitable (S108). For example, in a case where the amplitude, as the feature value of the muscle potential, is lower than the muscle potential for determination, the determination unit 46 determines that the worn position of the authentication terminal 10 is not suitable (No in S108). In this case, using any one of an image, audio, and vibration, the determination unit 46 causes the reporting unit 22 to report that the worn position of the authentication terminal 10 is unsuitable (S110). Accordingly, the target person OP that wears the authentication terminal 10 changes the worn position of the authentication terminal 10. Thereafter, the acquisition unit 44 and the determination unit 46 again perform processing in Step S104 and processing in each of the subsequent steps on the authentication terminal 10 of which the worn position is changed.

On the other hand, in a case where the amplitude, as the feature value of the muscle potential, is higher than the muscle potential for determination, the determination unit 46 determines that the worn position of the authentication terminal 10 is suitable (Yes in S108). In this case, the acquisition unit 44 acquires the measurement potential information from the first electrode 14 and the second electrode 16, and outputs the acquired measurement potential information to the determination unit 46 (S112).

The determination unit 46 corrects the muscle potential that is indicated by the muscle potential information (S116). For example, the determination unit 46 multiplies the muscle potential by the correction coefficient that is determined in advance, and make a correction by making the scale adjustment. Based on the corrected muscle potential and the measurement potential that is indicated by the measurement potential information, the determination unit 46 calculates the cardiac potential (S118). For example, the determination unit 46 uniformly removes the corrected muscle potential from the measurement potential, and thus calculates the cardiac potential. The determination unit 46 extracts one, or two or more feature values of the cardiac potential from the calculated cardiac potential (S120). The feature value of the cardiac potential, for example, is amplitude, a wave width, an inter-wave interval, and the like of each of the P wave, the Q wave, the R wave, the S wave, the T wave, and the U wave.

The determination unit 46 determines whether or not the target person OP who wears the authentication terminal 10 is a registrant (S122). For example, based on the extracted feature value of the cardiac potential and the cardiac potential information in the registrant database 52, the determination unit 46 determines whether or not the target person OP is a registrant. In a case where the cardiac potential information that is consistent with the extracted feature value of the cardiac potential is not registered in the registrant database 52, the determination unit 46 determines that the target person OP is not a registrant (No in S122), and determines whether or not the number of times that it is determined that the target person OP is a registrant is a prescribed number of times (S124). The prescribed number of times may be set in advance and may be stored, as one portion of the numerical data 54, in the terminal-side storage unit 42. When it is determined that the number of times of determination does not reach the prescribed number of times (No in S124), the determination unit 46 repeats Step S112 and subsequent steps. Moreover, in a case where the number of times of determination does not reach the prescribed number of times, the determination unit 46 may repeat Step S104 and subsequent steps. Accordingly, the acquisition unit 44 and the determination unit 46 acquire the measurement potential information multiple times, and based on a plurality of pieces of measurement potential information, determine whether or not the target person OP is a registrant. Because of this, the registrant can be suppressed from not being authenticated.

When it is determined that the number of times of determination reaches the prescribed number of times (Yes in S124), the determination unit 46 causes the reporting unit 22 to report that the target person OP is not registered in the registrant database 52, using any one of an image, audio, and vibration (S126), and the terminal-side processing unit 40 ends the authentication processing.

In a case where the extracted feature value of the cardiac potential is registered in the registrant database 52, the determination unit 46 determines that the target person OP is a registrant (Yes in S122), and transmits the authentication information that the target person OP is a registrant, to the control device 32 (S128). The determination unit 46 causes the reporting unit 22 to report that the target person OP is a registrant, using any one of an image, audio, and vibration (S130). Moreover, the determination unit 46 may cause the reporting unit 22 to report the health state of the target person OP relating to the pulse wave or the like, based on the information that the target person OP is a registrant, the cardiac potential and the muscle potential, and the like, and may output the health state to the control device 32, along with the authentication information. Accordingly, the terminal-side processing unit 40 ends the authentication processing.

In the control device 32, during the control processing, the apparatus-side processing unit 62 determines whether or not the authentication information that the target person OP is a registrant is acquired from the authentication terminal 10 (S150). The apparatus-side processing unit 62 is in the standby state until the authentication information is acquired from the authentication terminal 10 (No in S150). When receiving from the authentication terminal 10 the information that the target person OP is a registrant (Yes in S150), the apparatus-side processing unit 62 controls the control target CO (S152). For example, the apparatus-side processing unit 62 unlocks a door lock of a vehicle, and controls an air conditioner according to prior registration, or causes a driving source, such as an engine. Additionally, in a case where the health state of the target person OP is acquired along with the authentication information, the apparatus-side processing unit 62 may control the air conditioner in accordance with the health state. Accordingly, the apparatus-side processing unit 62 ends the control processing.

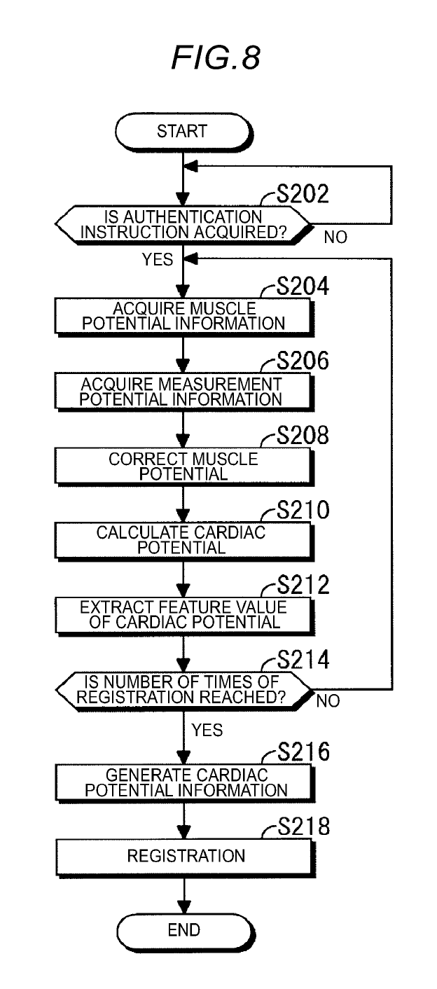

FIG. 8 is a flowchart illustrating an example of the registration processing that is performed by the authentication terminal 10. The authentication terminal 10 reads the registration and authentication program 50 and thus performs the registration processing. Among steps of the registration processing, a description of a step that is the same as the step of the authentication processing is simplified.

As illustrated in FIG. 8, in the registration processing, the acquisition unit 44 determines whether or not the registration instruction is acquired (S202). For example, when the target person OP inputs the registration instruction using the input unit 24, the acquisition unit 44 may determine that the registration instruction is acquired. The acquisition unit 44 is in the standby state until the registration instruction is acquired (No in S202). When acquiring the registration instruction (Yes in S202), the acquisition unit 44 causes the light emitting unit 18 to emit the measurement light, acquires muscle potential information from the light receiving unit 20, and outputs the acquired muscle potential information to the registration unit 48 (S204). The acquisition unit 44 acquires the measurement potential information from the first electrode 14 and the second electrode 16, and outputs the acquired measurement potential information to the registration unit 48 (S206).

The registration unit 48 corrects the muscle potential that is indicated by the muscle potential information, by making, for example, the scale adjustment that uses the correction coefficient (S208). The registration unit 48 removes the corrected muscle potential from the measurement potential that is indicated by the measurement potential information, and calculates the cardiac potential (S210), and extracts one feature value or a set of two or more feature values of the cardiac potential (S212). The registration unit 48 determines whether or not the number of times that the feature value is extracted is the number of times of registration (S214). The number of times of registration may be stored, as one portion of the numerical data 54 that is set in advance, in the terminal-side storage unit 42. When it is determined that the number of times of extraction is not the number of times of registration (No in S214), the registration unit 48 repeats Step S204 and subsequent steps, and repeats the extraction of the feature value of the cardiac potential. Accordingly, the registration unit 48 extracts sets of feature values, of which the number is the same as the number of times of registration.

When it is determined that the number of times of extraction reaches the number of times of registration (Yes in S214), the registration unit 48 generates one piece of, or two or more pieces of cardiac potential information that are registered in the registrant database 52, from one feature or a set of two or more feature values that are extracted (S216). For example, the registration unit 48 may generate an average value of each feature value as the cardiac potential information. Furthermore, the registration unit 48 may exclude an outlier or an abnormal value or the like from a plurality of feature values, and then may generate an average value of the calculated feature value as the cardiac potential information. The registration unit 48 registers one piece of, or two or more pieces of cardiac potential information in the registrant database 52 in a state of being associated with a new registrant ID (S218). Accordingly, the terminal-side processing unit 40 ends the registration processing.

As described above, in the authentication terminal 10, the first electrode 14 and the second electrode 16 measure the measurement potential including the cardiac potential and generate the measurement potential information, and the light emitting unit 18 and the light receiving unit 20 measure the muscle potential and generate the muscle potential information. In this manner, the light emitting unit 18 and the light receiving unit 20 for measuring the muscle potential are provided separately from the electrodes 14 and 16, and thus the authentication terminal 10 can improve the precision of the measurement of the muscle potential. Accordingly, although the state (for example, while in a walking or stopping state) of the target person OP changes, the authentication terminal 10 can measure the muscle potential that is close to actual muscle potential. As a result, the authentication terminal 10 removes high-precision muscle potential from the measurement potential and calculates the cardiac potential, compared with a case where regardless of the state of the target person OP, the muscle potential is uniformly removed from the measurement potential using a band pass filter. Because of this, the authentication terminal 10 can calculate the high-precision cardiac potential and thus can improve the precision of the authentication of the registrant using the cardiac potential.

Furthermore, the authentication terminal 10 calculates the cardiac potential based on the high-precision muscle potential regardless of the state of the target person OP. Therefore, the authentication terminal 10 can calculate the high-precision cardiac potential that is not influenced by the state of the target person OP. Because of this, one set of pieces of cardiac potential information on each registrant has to be registered in the registrant database 52. Accordingly, the authentication terminal 10 can reduce an amount of data in the registrant database 52 compared with a case where a plurality of sets of cardiac potential information that correspond to a plurality of states of the target person OP are registered as in the authentication terminal that has the band pass filter. Additionally, the authentication terminal 10 can compare one set of pieces of cardiac potential information that corresponds to each registrant and a set of measured feature values of the cardiac potential and thus can perform the authentication, without the need to compare the plurality of sets of pieces of cardiac potential information that correspond to the plurality of states and the measured feature value of the cardiac potential. Because of this, the time required for the authentication can be shortened.

In the authentication terminal 10, the first electrode 14 is provided inside of the casing 12 in the shape of a ring, and the second electrode 16 is provided outside of the casing 12. Accordingly, the first electrode 14 makes contact with one arm Ar (for example, the vicinity of the wrist) of the target person OP, and the second electrode 16 makes contact with the finger Fg of the other arm of the target person OP. Because of this, the authentication terminal 10 can increase a difference between a path from the first electrode 14 and heart of the target person OP and a path from the second electrode 16 to the heart of the target person OP. As a result, the authentication terminal 10 can improve the precision of the measurement of the cardiac potential regardless of a position of the heart of the target person OP.

Based on the muscle potential and the muscle potential for determination, the authentication terminal 10 determines whether or not the worn position of the authentication terminal 10 is suitable. In a case where the authentication terminal 10 determines that the worn position is suitable, the authentication terminal 10 can determine whether or not the target person OP is a registrant, based on the cardiac potential, and can generate the authentication information. Furthermore, in a case where the worn position is unsuitable, the authentication terminal 10 can report to the target person OP that the worn position is unsuitable and can provide a guidance so that the worn position becomes suitable, without determining whether or not the target person OP is a registrant. In this manner, the authentication terminal 10 determines the target person OP based on the cardiac potential in a case where the authentication terminal 10 is worn on a suitable position and performs the authentication. Because of this, the authentication terminal 10 can improve the precision for the authentication. Additionally, the authentication terminal 10 determines the worn position based on the muscle potential. Because of this, the authentication terminal 10 can improve the precision of the determination of the worn position compared with a case where the worn position is determined with the measurement potential in which noise increases because a plurality of potentials are included such as the muscle potential and the cardiac potential.

The authentication terminal 10 removes the muscle potential that results from correcting the amplitude of the muscle potential, from the measurement potential, and calculates the cardiac potential. Accordingly, the authentication terminal 10 can associate a scale of the muscle potential that is measured by the light receiving unit 20, with a scale of the measurement potential that includes the cardiac potentials which are measured in the electrodes 14 and 16. Because of this, the authentication terminal 10 can improve the precision of the calculation of the cardiac potential.

In the authentication terminal 10, the light emitting unit 18 emits blue light, as the measurement light for measuring the muscle potential, to the target person OP. Accordingly, the authentication terminal 10 reduces an influence on a capillary vessel or the like within the body in a case where light with a long wavelength, such as green light, is emitted, and thus can measure the high-precision muscle potential from the surface of the body of the target person OP.

The authentication terminal 10 determines whether or not the target person OP is a registrant, based on the cardiac potential and the registrant database 52, and outputs the authentication information, which is generated based on a result of the determination, to the control device 32. Accordingly, the authentication terminal 10 can reduce an amount of data in the authentication information compared with a case where the cardiac potential or the feature value of the cardiac potential is transmitted as the authentication information to the control device 32.

A function of a configuration, a connection relationship, the number of constituent elements, a shape, an arrangement, and the like in the embodiment described above are properly changed, deleted, and so on within the scope of the invention and the scope that is equivalent to the scope of the invention. The order of steps according to the embodiment may be properly changed.

In the above-described embodiment, as an example, the authentication system 30 in which the control device 32 is mounted in a vehicle or the like is described, but the authentication system 30 is not limited to this. For example, the authentication system 30 may find application in an authentication system that includes a control device which locks or unlocks a key to a door of a house.

In the above-described embodiment, the authentication terminal 10 sets the result of the authentication, which is obtained by determining whether or not the target person OP is a registrant based on the feature value of the cardiac potential and the cardiac potential information in the registrant database 52, to be the authentication information, but the authentication information is not limited to this. For example, the authentication terminal 10 may generate the feature value of the cardiac potential as the authentication information, and may transmit the resulting authentication information to the control device 32. In this case, the control device 32 may have the registrant database 52, and may determine whether or not the target person OP is a registrant, based on the feature value of the cardiac potential that is indicated by the authentication information which is acquired from the authentication terminal 10, and on the registrant database 52.

In the above-described embodiment, as an example, the authentication terminal 10 that determines whether or not the target person OP is a registrant, using the cardiac potential, but the authentication terminal 10 may determine whether or not the target person OP is a registrant, using any other piece of information in addition to the cardiac potential, and thus may improve the robustness of the authentication. For example, the authentication terminal 10 may output as the authentication information the result of the determination that is obtained by determining whether or not the target person OP is a registrant who is registered in the registrant database that is set in advance, based on any one of authentication that uses a fingerprint, authentication that uses a finger vein, authentication that uses a gait which is detected by an acceleration sensor, and authentication that uses an iris, in addition to the authentication that uses the cardiac potential. Accordingly, the authentication terminal 10 can improve the robustness of the authentication. Additionally, the authentication that uses a fingerprint may be performed along with the measurement of the measurement potential, with the second electrode 16 as a transparent electrode.

An authentication terminal according to an aspect of this disclosure includes: a casing that is worn on a body of a target person; first and second electrodes that are provided in the casing and measure a measurement potential as biological information; a light emitting unit that is provided in the casing and emits measurement light for measuring a muscle potential to the body of the target person; a light receiving unit that receives the measurement light and outputs muscle potential information which corresponds to the muscle potential; and a terminal-side control unit that outputs authentication information which is generated based on the muscle potential that is indicated by the muscle potential information, and on a cardiac potential that is calculated from the measurement potential between the first electrode and the second electrode to another device.

In the authentication terminal according to the aspect of this disclosure, the first electrode and the second electrode measure the measurement potential including the cardiac potential and the muscle potential is measured using the light emitting unit and the light receiving unit. In this manner, the authentication terminal that includes the light emitting unit and the light receiving unit, separately from the electrodes, and thus can reduce an influence due to a state of the target person and can improve the precision of the measurement of the muscle potential. Accordingly, the authentication terminal can improve the precision of the cardiac potential that is calculated from the muscle potential and the measurement potential, and can improve the precision of the authentication of the registrant, which uses the cardiac potential.

In the authentication terminal according to the aspect of this disclosure, the casing may be in the shape of a ring, the first electrode may be provided inside of the casing, the second electrode may be provided outside of the casing, and the measurement potential may be measured by causing one portion of the body of the target person to make contact with both of the first electrode and the second electrode.

With this configuration, in the authentication terminal according to the aspect of this disclosure, the first electrode makes contact with one arm or the like of the target person, and the second electrode makes contact with the finger of the other arm of the target person. Because of this, a difference between a path from the first electrode and the heart and a path from the second electrode to the heart increases. As a result, the authentication terminal can improve the precision of the measurement of the cardiac potential.

In the authentication terminal according to the aspect of this disclosure, based on the muscle potential that is indicated by the muscle potential information, and on a muscle potential for determination, which is registered in advance by the target person, the terminal-side control unit may determine whether or not a worn position of the authentication terminal is suitable, and the terminal-side control unit may generate the authentication information in a case where the worn position is determined as suitable, and may report to the target person that the worn position is unsuitable, in a case where the worn position is determined as unsuitable.

In this manner, the authentication terminal according to the aspect of this disclosure determines whether or not the worn position of the authentication terminal is suitable, based on the muscle potential that has the high precision of the calculation and the muscle potential for determination. The authentication terminal generates the authentication information in a case where the worn position is suitable, and makes a report in a case where the worn position is unsuitable. With this configuration, with the determination that is based on the cardiac potential that is measured in a suitable worn position, the authentication terminal can further improve the authentication information.

In the authentication terminal according to the aspect of this disclosure, the terminal-side control unit may remove the muscle potential that results from correcting an amplitude of the muscle potential that is indicated by the muscle potential information, from the measurement potential, and may calculate the cardiac potential.

With this configuration, the authentication terminal according to the aspect of this disclosure can associate a scale of the muscle potential that is measured by the light receiving unit, with a scale of the measurement potential that includes the cardiac potentials which are measured in the electrodes. Because of this, the authentication terminal can improve the precision of the calculation of the cardiac potential.

In the authentication terminal according to the aspect of this disclosure, the light emitting unit may emit blue light as the measurement light.

With this configuration, the authentication terminal according to this disclosure can measure the high-precision muscle potential, without being influenced by a capillary vessel or the like within the body.

In the authentication terminal according to the aspect of this disclosure, the terminal-side control unit may output, as the authentication information, a result of the determination that is obtained by determining whether or not the target person is registered in a registrant database that is set in advance based on the cardiac potential.

With this configuration, the authentication terminal according to the aspect of this disclosure can reduce an amount of data in the authentication information that is output.

In the authentication terminal according to the aspect of this disclosure, the terminal-side control unit may output, as the authentication information, the result of the determination that is obtained by determining whether or not the target person is registered in the registrant database that is set in advance based on a fingerprint or a finger vein.

With this configuration, the authentication terminal according to the aspect of this disclosure can improve the robustness of the authentication.

The principles, preferred embodiment and mode of operation of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. An authentication terminal comprising:
a casing that is worn on a body of a target person;
first and second electrodes that are provided in the casing and measure a measurement potential as biological information;
a light emitting unit that is provided in the casing and emits measurement light for measuring a muscle potential to the body of the target person;
a light receiving unit that receives the measurement light and outputs muscle potential information which corresponds to the muscle potential; and
a terminal-side control unit that outputs authentication information which is generated based on the muscle potential that is indicated by the muscle potential information, and on a cardiac potential that is calculated from the measurement potential between the first electrode and the second electrode to another device.

2. The authentication terminal according to claim 1,
wherein the casing is in the shape of a ring,
the first electrode is provided inside of the casing,
the second electrode is provided outside of the casing, and
the measurement potential is measured by causing one portion of the body of the target person to make contact with both of the first electrode and the second electrode.

3. The authentication terminal according to claim 1,
wherein, based on the muscle potential that is indicated by the muscle potential information, and on a muscle potential for determination, which is registered in advance by the target person, the terminal-side control unit determines whether or not a worn position of the authentication terminal is suitable, and
the terminal-side control unit generates the authentication information in a case where the worn position is determined as suitable, and reports to the target person that the worn position is unsuitable, in a case where the worn position is determined as unsuitable.

4. The authentication terminal according to claim 1,
wherein the terminal-side control unit removes the muscle potential that results from correcting an amplitude of the muscle potential that is indicated by the muscle potential information, from the measurement potential, and calculates the cardiac potential.

5. The authentication terminal according to claim 1,
wherein the light emitting unit emits blue light as the measurement light.

6. The authentication terminal according to claim 1,
wherein, the terminal-side control unit outputs, as the authentication information, a result of the determination that is obtained by determining whether or not the target person is registered in a registrant database that is set in advance based on the cardiac potential.

7. The authentication terminal according to claim 6,
wherein the terminal-side control unit outputs, as the authentication information, the result of the determination that is obtained by determining whether or not the target person is registered in the registrant database that is set in advance based on a fingerprint or a finger vein.
